(19) United States
(12) Patent Application Publication
(10) Pub. No.: US 2025/0302722 A1
(43) Pub. Date: Oct. 2, 2025

(54) FRAGRANCE RELEASE MECHANISM, METHOD AND USES THEREOF

(71) Applicant: UNIVERSIDADE DO MINHO, Braga (PT)

(72) Inventors: Artur Manuel CAVACO PAULO, Braga (PT); Filipa Daniela GOMES GONÇALVES, Braga (PT); Artur Jorge ARAÚJO MAGALHÃES RIBEIRO, Braga (PT); Carla Manuela PEREIRA MARINHO DA SILVA, Andar (PT)

(73) Assignee: UNIVERSIDADE DO MINHO, Braga (PT)

(21) Appl. No.: 19/238,668

(22) Filed: Jun. 16, 2025

Related U.S. Application Data
(63) Continuation of application No. 18/520,428, filed on Nov. 27, 2023, now Pat. No. 12,329,841.
Application No. 18/520,428 is a continuation of application No. 18/003,127, filed on Dec. 22, 2022.

(30) Foreign Application Priority Data
Jul. 3, 2020 (PT) 116561
Nov. 6, 2020 (EP) 20206292.3

(51) Int. Cl. A61K 8/64; A61K 8/11; A61Q 15/00

(52) U.S. Cl. CPC A61K 8/64; A61K 8/11; A61Q 15/00

(57) ABSTRACT

The present disclosure relates to a release composition comprising a protein and an active agent, wherein the active agent is released when in presence of an electrolyte solution. A kit and an article comprising the release composition of the present-subject matter are also encompassed.

### CROSS-REFERENCE

The present application is a continuation of U.S. patent application Ser. No. 18/520,428, filed Nov. 27, 2023, which is a continuation of U.S. patent application Ser. No. 18/003,127, filed Dec. 22, 2022, which is a § 371 U.S. National Stage Entry of International Application No. PCT/IB2021/056011, filed Jul. 5, 2021, which claims the benefit of Portuguese Application No. 116561, filed Jul. 3, 2020, and European Application No. 20206292.3, filed Nov. 6, 2020, each of which are hereby incorporated by reference in their entirety herein.

### SEQUENCE LISTING

The instant application contains a Sequence Listing which has been submitted electronically in ASCII format and is hereby incorporated by reference in its entirety. Said ASCII copy, created Dec. 13, 2023, is named 10224_013064-US2_Sequence Listing.txt and is 32,000 bytes in size.

### TECHNICAL FIELD

The present disclosure relates to a mechanism of adsorption and dissociation involving fragrance molecule and an odorant-binding protein (OBP-I) regulated simultaneously by human sweat and temperature. This system can be used in cosmetic formulations where the fragrances are released upon presence of sweat at body temperature.

### BACKGROUND

Odorant-binding proteins (OBPs) are small water-soluble proteins, belonging to lipocalins superfamily.¹⁻² They are responsible to transport hydrophobic odorous molecules, called odorants, in their calyx-shaped cavity, across the aqueous mucus barrier towards the olfactory receptors, where a cascade of transduction signal is traduced in the brain's interpretation.³⁻⁴ These proteins are also described as involved in removing odorants from the olfactory receptors after their stimulation.⁵⁻⁶

Several mammalian odorant-binding proteins have been identified and some of them isolated from nasal mucus such as bovine, pig, boar, panda, mice, rats and humans.⁷⁻¹³

DNA sequences of mammalian OBPs present low similarity: porcine OBP (OBP-I) and human OBP (hOBP_{IIa}) present only 13.9% of DNA sequence similarity; OBP-I and bovine OBP present 42.7%. 4 Despite wide genetic variability between OBPs from different mammalian species, lipocalin members present few characteristic signatures that allow their identification as the case of the conserved tertiary structure, presenting a 6-barrel structure composed by eight β-strands linked by seven loops and connected to a short α-helix close to the C-terminus and a ninth β-strand followed by the disordered C-terminal tail.¹⁴⁻¹⁶ The structure of OBPs is highly stable and resistant to degradation by temperature, organic solvents, pH variation, or proteolytic digestion.^{17-18 19} The FT-IR spectra for porcine OBP revealed a structure exceptionally stable to thermal denaturation (up to 80° C.), particularly in the presence of a ligand.²⁰ Furthermore, vertebrate OBPs show capacity to reverse the unfolding of protein even after denaturation.²⁰

Human body produces unpleasant odours associated with stress, anxiousness, nervousness and physical exercise.²¹ To prevent or reduce their occurrence, antibacterial agents and fragrances are commonly added to cosmetic formulations. However, drawbacks related with the limited effect against different odours and with the residual amount of these deodorants are detected in clothing and skin.²²

OBPs have affinity for several molecules associated with odorific feeling. All those molecules are volatile and detected by OBPs at very low concentrations, being a system highly sensitive. The fast responsive time of the OBPs and the high stability of these proteins create an excellent biological element as biosensor for detection of the dangerous substances and pathogens, pesticides and drugs present in food or water^{18, 23} as well as the potential use as deodorizer and medical diagnostics.²⁴⁻²⁵

Odorific molecules can be associated with pleasant or unpleasant feelings. OBP have affinities for all molecules associated with odors.³⁻⁴ Fragrances are compositions containing odorific molecules with pleasant feeling.

The use of 1-aminoanthracene (1-AMA) as odour model molecule provides a capacity to measure the binding capacity of odorant-binding protein, by fluorescence assay. The free 1-AMA and 1-AMA bound to pig OBP-I can be quantified measuring the fluorescence with excitation wavelength at 295 nm. The maximum wavelength of 1-AMA bound to OBP-I is shifted from 537 nm to 481 nm.²⁰ The non-fluorescence odorant can be measured by competitive assays or by gas chromatography-mass spectrometry.

The following works already reported the interaction between OBPs and odour model molecules, as well as with lipidic structures such as liposomes.

Filipa Gonçalves et al (2018) “Two Engineered OBPs with opposite temperature-dependent affinities towards 1-aminoanthracene” mentions two recombinant proteins based on pig OBP sequence (i) truncated OBP (tOBP-F44A/F66A) obtained from the deletion of the first 16 residues of the N-terminal and the replacement of two phenylalanine residues at the binding pocket by alanine residues (F44A and F66A), and (ii) OBP::GQ₂₀::SP-DS3 resulted of the fusion of OBP-I with a spacer of 20 repetitions of glycine-glutamine residues and the anchor peptide SP-DS3.³⁴ Experimental and molecular modelling data showed that 1-AMA model ligand binds preferentially to tOBP-F44A/F66A at 25° C. while ligand binds to OBP::GQ₂₀::SP-DS3 favourably at 37° C.³⁴

Filipa Gonçalves et al (2018) “OBP fused with cell-penetrating peptides promotes liposomal transduction” report the fusion of porcine OBP with cell-penetrating peptides (CPPs, e.g. TAT, Pep-1 and pVEC). The study revealed different efficiencies on 1-AMA transduction into liposomes.³⁰

Filipa Gonçalves et al (2018) “1-Aminoanthracene Transduction into Liposomes Driven by Odorant-Binding Protein Proximity” discloses the design of two fusion proteins based on pig OBP fused with anchor peptide SP-DS332 in absence and presence of a spacer (GQ₂₀). This work demonstrated that the 1-AMA transduction into liposomes is driven by proximity of protein anchored to liposomal membrane (advantage for absence of spacer).³³

Filipa Gonçalves et al (2019) “Release of Fragrances from Cotton Functionalized with Carbohydrate-Binding Module Proteins” discloses the design of fusion protein based on pig OBP fused with a spacer (GQ₂₀) and a carbohydrate binding module (CBM). The work demonstrated the affinity of protein to one fragrance (β-citronellol) and the release of this fragrance from cotton in presence of sweat.²⁵ Regardless, the release capacity in the presence of sweat is inferior as compared to the native OBP.

Alessandro Capo et al (2018) “The porcine odorant-binding protein as molecular probe for benzene detection” discloses pig odorant-binding protein to be used as probe for benzene detection in atmosphere, through fluorescence assay.²⁸

Nunzio Cennamo et al (2015) “Easy to Use Plastic Optical Fiber-Based Biosensor for Detection of Butanal” reports the detection of butanal (20-1000 μM) by porcine odorant-binding protein through competitive assay. This aldehyde is very toxic, exhibiting high risks for human health like cytotoxicity and cancer. The authors describe an optical biosensor to detect butanol in liquid samples.²⁷

Carla Silva et al (2014) “Odorant binding proteins: a biotechnological tool for odour control” discloses the application of porcine odorant-binding protein for release of fragrances from a cotton fabric to mask smoke odour. The authors confirmed the functionalization of OBP on fabrics. They tested only the release of one fragrance from textile. Contrary to the work of Silva et al., the present disclosure includes the release mechanism of different fragrances or other molecules as response of human perspiration with upper efficiency. Additionally, the subject-matter of the present disclosure is suitable for use in textile and cosmetic fields.

Paolo Pelosi et al (2014) “Structure and biotechnological applications of odorant-binding proteins” discloses the possibility of OBPs to be used as a sensor to detect volatile and slow release of odorant molecules.

Lei Han et al (2014) “Operating Mechanism and Molecular Dynamics of Pheromone-Binding Protein ASP1 as Influenced by pH” indicates pheromone-binding protein ASP1 as binds odorants at low pH and the dissociation respond to pH change. The authors describe the benefit of this research in biotechnology and agriculture. The results were determined by molecular docking and dynamics simulations.

Alberto Mazzini et al (2007) “Dissociation and unfolding of bovine odorant binding protein at acidic pH” discloses the structure of bovine OBP at neutral and acidic pH, by molecular simulation.

Mariella Parisi et al (2003) “Unfolding and refolding of porcine odorant binding protein in guanidinium hydrochloride: equilibrium studies at neutral pH” discloses the denaturant effect of guanidinium hydrochloride, a well-known chaotropic agent, in folding/unfolding of protein. The aim of this fundamental study was to understand the structure of the OBP protein, in particular its unfolding and refolding process.

Document WO 0123890A1 discloses a detector array based on sensing elements within a solid support with use for clinical samples or cell extracts, in gaseous state. It is an immunoassay utilizing viral peptides.

Document EP0335654A3 discloses the method for gene isolation of odorant-binding protein from rat and a protein production method.

Document WO2001012806A3 described OBPII as a fixer of hydrophobic ligands such as odours that can be used for personal hygiene, agri-food system and nutritional and therapeutic uses.

These facts are disclosed in order to illustrate the technical problem addressed by the present disclosure.

### General Description

The present disclosure is related to adsorption and dissociation mechanism of native odorant-binding protein (OBP-I), in particular pig OBP-I (SEQ. ID NO. 1), and OBP fused with linker GQ 20× and KP peptide (OBP::GQ₂₀::KP, SEQ. ID NO. 21). These proteins have a negative charge of approximately-20 (pH 7.4) due to the high content in aspartic acid and glutamic acid residues. The isoelectric point value of these proteins are between 4.08 and 4.65.26

In an embodiment, the solution here disclosed can have a high impact in human social life that are associated with perspiration issues. The system has several advantages including the use of bioinspired cosmetic bioingredients (green solutions) without damaging for the ecosystems.

The mechanism here disclosed divulge that odorant-binding protein, in particular porcine odorant-binding protein (OBP-I), presents high affinity (adsorption) to fragrances in 50 mM Tris-HCl pH 7.5 buffer, at 37° C. The affinity constant (Ka) of OBP-I was of 4.00±0.03 μM. On the other hand, OBP-I presents a reverse mechanism, i.e., the dissociation of fragrance from its binding pocket with reduced Ka (0.20±0.02 UM) when in exposition of perspiration (sweat), even at different pH (range of 4.0-8.5, Table 2). Similarity, OBP::GQ₂₀::KP presents high affinity to fragrances in buffer, at 37° C. (Ka=4.00±0.04 μM) that is reduced in presence of an electrolyte solution, such as sweat (Ka=0.59±0.01 μM). Therefore, OBPs presents reduced affinity when in contact with perspiration, releasing the fragrance in this condition.

Surprisingly, OBP::GQ₂₀::KP (SEQ ID NO. 21) showed 6.8× more fragrance release in presence of sweat versus the presence of buffer. These values are superior to the values reported in state of art, in particular to the values reported for OBP::GQ₂₀::CBM (SEQ ID NO. 22), where the release mechanism showed 1.3× release of fragrance in presence of sweat. 25

The adsorption and dissociation mechanism of porcine odorant-binding protein can be done in a repeated manner.

Human sweat can be used as a trigger to release/dissociate a fragrance from OBP-I. Therefore, the subject-matter of the present disclosure can be used in skin care products as well as in textile items, in particular clothes.

In an embodiment, the present disclosure relates to a protein with an amino acid sequence similar to mammalians odorant-binding proteins to be incorporated in formulations for cosmetic or textile applications.

In an embodiment, the native form of odorant-binding protein may be from pig, human, dog, cat, rat, mouse, cow, boar, panda, Chinese hamster, Meishan pig, Guinea pig, Tibetan pig, horse, dolphin and chimpanzee.

In an aspect of the present disclosure, applications of the present subject-matter may be based on the release of odorant molecules from odorant-binding protein, triggered by electrolyte solutions at body temperature.

In an embodiment, the electrolyte solution refers to a solution with a NaCl concentration higher than 9.5 grams/L, in particular to a solution with a NaCl concentration ranging from 9.5 to 45 grams/L. Preferably, the electrolyte solution is human sweat, pet sweat, salty water or micellar water.

In an embodiment, human sweat may comprise water, lactic acid, urea and minerals, such as sodium, potassium, calcium, and magnesium.

In an embodiment, cosmetic applications may be for skin and hair care. Skin care applications may be related with OBPs formulated in specialty formulations for skin creams, lipsticks, lips creams and face mask powders, face and body creams, skin clarifiers, primers and foundations.

In a further embodiment, hair care applications may be related with OBPs formulated in eyelash mascaras, hair shampoos, hair serum, hairs masks, hair conditioners, or hair coloration creams.

The present disclosure relates to a release composition comprising an isolated or artificial protein with at least 90% of identity with an amino acid sequence selected from the following list: SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15. SEQ. ID NO. 16, SEQ. ID NO. 17, SEQ. ID NO. 18, SEQ. ID NO. 19, SEQ. ID NO. 20, SEQ. ID NO. 21, or mixtures thereof, preferably 91% identical, 92% identical, 93% identical, 94% identical, 95% identical, 96% identical, 97% identical, 98% identical, 99% identical or identical; an active agent selected from a list comprising a deodorizing agent, a natural essence, a fragrance, a moisturizing agent, or mixtures thereof; wherein the active agent is bounded to the protein; and wherein the protein releases the active agent in the presence of an electrolyte solution, at a temperature between 10-60° C.

In an embodiment, the release composition comprises an isolated or artificial unmodified protein with at least 90% of identity with an amino acid sequence selected from the following list: SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15. SEQ. ID NO. 16, SEQ. ID NO. 17, SEQ. ID NO. 18, SEQ. ID NO. 19, SEQ. ID NO. 20, SEQ. ID NO. 21, or mixtures thereof, preferably 91% identical, 92% identical, 93% identical, 94% identical, 95% identical, 96% identical, 97% identical, 98% identical, 99% identical or identical.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48:443-453) to find the global (over the whole the sequence) alignment of two sequences that maximizes the number of matches and minimizes the number of gaps. The BLAST algorithm (Altschul et al. (1990) J Mol Biol 215:403-10) calculates percent sequence identity and performs a statistical analysis of the similarity between the two sequences. The software for performing BLAST analysis is publicly available through the National Centre for Biotechnology Information (NCBI). Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. The sequence identity values, which are indicated in the present subject matter as a percentage were determined over the entire amino acid sequence, using BLAST with the default parameters.

In an embodiment, the composition of the present subject-matter comprises 0.01 to 5000 μM of the unmodified protein.

In an embodiment, the composition of the present subject-matter comprises 0.01 to 5000 UM of the protein.

In another embodiment, the release composition comprises 0.1 μM to 2 M of active agent, preferably 0.2 μM to 1 M.

In an embodiment, the protein has an affinity constant of 1-4.5 μM to the active agent, in water or buffer solutions, preferably Tris-HCl, phosphate solution, and/or phosphate buffered saline. In a further embodiment, the affinity constant of the protein to the active agent ranges between 0.1-0.99 μM in the electrolyte solution, preferably in sweat.

The present disclosure relates to a fragrance release composition comprising: 0.01 to 5000 UM of an isolated or artificial protein with at least 90% of identity with an amino acid sequence selected from the following list: SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15. SEQ. ID NO. 16, SEQ. ID NO. 17, SEQ. ID NO. 18, SEQ. ID NO. 19, SEQ. ID NO. 20, SEQ. ID NO. 21 or mixtures thereof; 0.1 μM to 2 M of an active agent selected from a list comprising a deodorizing agent, a natural essence, a fragrance, a moisturizing agent, or mixtures thereof; wherein the active agent is bounded and/or entrapped to the protein; and wherein the protein releases the active agent in the presence of an electrolyte solution, at a temperature between 10-70° C., preferably 10-60° C.; wherein the affinity constant of the protein to the active agent, in water, ranges between 1-4.5 μM; wherein the active agent has a molecular weight from 20 to 1000 g/mol; wherein the electrolyte solution is sweat, salty water or micellar water.

In an embodiment, the active agent has a molecular weight between 20 to 1000 g/mol, preferably 75-300 g/mol. In a further embodiment, the active agent is a fragrance molecule. In a yet further embodiment, the bioactive agent comprises a functional group selected from aromatic, aldehyde or alcohols. In a yet further embodiment, the active agent is a fragrance molecule, selected from a list comprising the molecules listed in Table 1.

Table 1—List of fragrances and their properties.

TABLE 1
List of fragrances and their properties.

| | | | | MW |
|---|---|---|---|---|
| CAS# | Name | Odor description | Chemical Family | (g/mol) |
| 85213-22-5 | 2-acetyl-1- | roasted/bread | ketone | 111.14 |
| | pyrroline | | | |
| 8000-41-7 | α-terpineol | lilac | alcohol/terpene | 154.25 |
| 502-99-8 | β-ocimene | sweet herbal | hydrocarbon/terpene | 136.24 |
| 140-11-4 | benzyl acetate | strawberry/pear | ester | 150.17 |
| 123-86-4 | butyl acetate | banana | ester | 116.16 |
| 76-22-2 | camphor | camphora | ketone/terpene | 152.24 |
| 6485-40-1 | carvone | mint | ketone/terpene | 150.22 |
| 5392-40-5 | citral | lemon/citrus | aldehyde/terpene | 152.24 |
| 106-22-9 | citronellol | citronella/rose-like | alcohol/terpene | 156.27 |
| 91-64-5 | coumarin | sweet | lactone/aromatic | 146.15 |
| | | vanilla/pleasant | | |
| 431-03-8 | diacetyl | buttery | ketone | 86.09 |
| 97-53-0 | eugenol | cloves | aromatic alcohol | 164.20 |
| 6413-10-1 | fructone | apple | ester | 174.19 |
| 706-14-9 | gamma | coconut | lactone | 170.25 |
| | decalactone | | | |
| 104-61-0 | gamma | peach/fruity | lactone | 156.23 |
| | nonalactone | | | |
| 106-24-1 | geraniol | floral/sweet rose | terpene | 154.24 |
| 24851-98-7 | hedione | floral/jasmine | ester | 226.32 |
| 123-92-2 | isoamyl acetate | pear/banana | ester | 130.19 |
| 67920-63-2 | lilac aldehyde | floral/lilac | aldehyde/terpene | 168.24 |
| 5989-27-5 | limonene | citric | terpene | 136.23 |
| 126-91-0 | linalool | lavender/bergamot | terpene | 154.25 |
| 55066-48-3 | mefrosol | floral/rose | alcohol | 178.27 |
| 2216-51-5 | menthol | peppermint | alcohol | 156.26 |
| 623-42-7 | methyl butyrate | apple/pineapple | ester | 102.13 |
| 123-35-3 | myrcene | herbal/woody | terpene | 136.24 |
| 80-56-8 | pinene | pine | terpene | 136.24 |
| 357650-26-1 | pomarose | plums/apples rose | ketone | 166.26 |
| 89-82-7 | pulegone | peppermint | ketone/terpene | 152.24 |
| 65113-99-7 | sandalore | sandalwood | alcohol | 210.36 |
| 121-33-5 | vanilin | vanilla | aldehyde/aromatic | 152.15 |

In an embodiment, the release of the active agent occurs during 30 seconds to 24 h.

In an embodiment, electrolyte solution has a pH between 4.0-8.5. In a further embodiment, the electrolyte solution is sweat, salty water or micellar water, preferably human sweat or pet sweat.

In an embodiment, the unmodified protein is stable in polar and non-polar solvents, including methanol, butanol, benzene, ethanol and undecanol as well as buffer solutions, preferably Tris-HCl, phosphate, or phosphate buffered saline (PBS)). In a further embodiment, the unmodified protein is also stable in temperatures between 18-70° C., preferably 18-60° C., and in a pH range of 4.0-10.0.

In an embodiment, the protein is stable in polar and non-polar solvents, including methanol, butanol, benzene and undecanol as well as buffer solutions (Tris-HCl, phosphate, PBS). In a further embodiment, the protein is also stable in temperatures between 18-70° C., preferably 18-60° C., and in a pH range of 4.0-10.0.

In an embodiment, the release of the active agent from the protein occurs at 20-40° C.

In an embodiment, the composition further comprises glycerol, erythritol, arabitol, sorbitol, mannitol, xylitol, mannisdomannitol, glucosylglycerol, glucose, fructose, sucrose, trehalose, isofluoroside, dextrans, levans, polyethylene glycol, salts of chloride, citrate, sulfates, acetate or phosphates, or mixtures thereof.

An aspect of the present disclosure comprises a kit or article comprising the composition described in the present subject-matter. In an embodiment, the article comprising said composition can be selected from a list comprising fabric, textiles, fibers, clothes, scarfs, hats, gloves, socks and turbans, shoes, insoles, bags, handbags, detergents, creams, lotions, foams, perfumes, softeners, aerosols, deodorants, lipsticks, lip creams, face mask powders, face and body creams, skin clarifiers, primers, foundations, hair shampoos, hair serum, hairs masks, hair conditioners or hair coloration creams.

The present disclosure also relates to the use of the fragrance release composition described in the present subject-matter in cosmetics, preferably as a cosmetic agent/composition, more preferably skin care or hair care; as well as the use of said composition in the textile industry.

The present disclosure also relates to the use of the fragrance release composition as a deodorant agent/composition.

### BRIEF DESCRIPTION OF THE DRAWINGS

The following FIGURES provide preferred embodiments for illustrating the disclosure and should not be seen as limiting the scope of invention.

FIG. 1: Schematic representation Sodium dodecyl sulfate and polyacrylamide (SDS-PAGE) gel electrophoresis under reducing conditions (A), and circular dichroism (CD) spectrum (B) of native pig odorant-binding protein. Mw: Precision Plus Protein™ standards (BioRad).

### DETAILED DESCRIPTION

The present disclosure concerns the method of manufacture of a fragrance/protein complex and the adsorption and dissociation mechanism of native odorant-binding protein (OBP-I), in particular pig OBP-I, and native protein fused with GQ₂₀ and KP peptide. This mechanism involves the release of active agents, preferably odorific molecules (or fragrances), in response to human perspiration.

The present disclosure presents high impact in textile and cosmetic fields, particularly in the release of fragrances from lotion and cream base products as well as textile fabrics, awarding a green solution.

In an embodiment, the produced odorant-binding protein and fragrance/protein complex are very soluble and stable at different solvents (like methanol, butanol, benzene and undecanol) and different range of temperature (18-60° C.) and different range of pH (4.0-10.0). 12, 27-29

In a further embodiment, the mixture of odorific molecules and the OBP-I or OBP::GQ₂₀::KP proteins showed improved results regarding the affinity constants in a buffer solution and in sweat (Table 2). In particular, the lower Ka in sweat enforces the use of OBP proteins when sweat-triggered responses are envisaged. Notably, the adsorption and dissociation mechanism by the proteins is reversible. In response to human perspiration at different range of pH (pH 4.0-8.5) the disclosed proteins released more than 20× to 6.8× (OBP-I and OBP::GQ₂₀::KP, respectively) of odorific molecules compared with other fusion proteins based on porcine odorant-binding protein (Table 2). Thus, the release of fragrance occurs in response of perspiration and it is independent of pH of sweat of each human. Additionally, the fragrance release occurs at least during 0.5-24 h.

In an embodiment, a variety of active agents, including deodorizing agents, natural essences, fragrance agents, moisturizing agents, or mixtures thereof, can be used in the complex formation, allowing a wide range of cosmetic uses. In particular, pleasant odorific molecules (of different size and shape) showed great affinity to the odorant-binding protein, in particular porcine odorant-binding protein.

In an embodiment, the odorific molecules used in the present disclosure belong to different functional groups (aromatic, aldehyde, alcohols). In a further embodiment the odorific molecules comprise molecules with a molecular weight ranging from 20,00 to 1000.00 g/mol, and the concentration varies from 0.2-2000 μM.

In an embodiment, odorific molecules have a molecular weight between 75 to 300 g/mol.

In an embodiment, the system efficiently responded to human perspiration, releasing fragrances over time. Importantly, the system did not respond against the water existent in human body, giving specificity and robustness to the subject-matter presented in this disclosure. Without this selectivity, i.e., if the system responded to water, the OBP protein would immediately release the odorific molecules when in contact with the skin.

### Example

In an embodiment, native porcine odorant-binding protein (OBP-I) and OBP-I fused with a spacer glycine-glutamine, repeated 20× (GQ₂₀) and with a carbohydrate-binding module (OBP::GQ₂₀::CBM) were cloned in plasmid pET28a and transformed into Escherichia coli BL21 (DE3). The proteins were expressed and purified through Nickel magnetic beads with specificity to His-tag present in the protein's N-terminal. 10 μM of protein were loaded on sodium dodecyl sulfate and polyacrylamide (SDS-PAGE) gel electrophoresis under reducing conditions. The same concentration was used to determine the structure of proteins by circular dichroism (CD) spectroscopy.

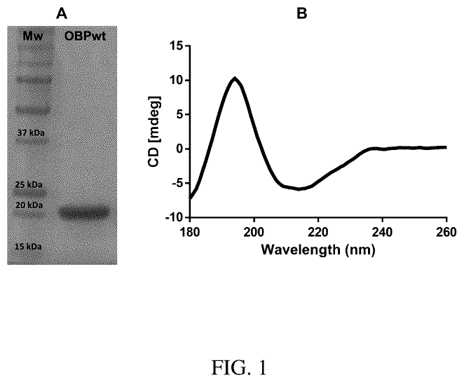

The protein purified in laboratory reveals a high level of purity (observed by SDS-PAGE gel, FIG. 1-A) and secondary structure in barrel, a consequence of the presence of a high number of β-sheets, as analysed by circular dichroism spectroscopy (FIG. 1-B).

In an embodiment, the pure odorant-binding protein was lyophilized and used in further procedures. To determine the affinity of the odorant-binding protein to odour molecules, 1-aminoanthracene (1-AMA) was used as an odour model molecule. Increased concentrations of fluorescent ligand model 1-aminoanthracene (1-AMA) were added to 1 μM of protein, and the formation of ligand/protein complex was quantified after 1 h at 37° C. by fluorescence emission at 481 nm (excitation at 295 nm).³⁰ Dissociation constant (Kd) was determined from a plot of fluorescence intensity versus concentration of ligand, obtained with a standard non-linear regression method, described in Malpeli et al. (1998).³¹ The affinity behaviour of protein in presence of sweat solution was performed through a competitive fluorescence assay. Here, 1 μM of protein was mixed with 2 μM of 1-AMA and incubated at 37° C. for 1 h. After this period, increased volumes of sweat solution were added to the complex and incubated at same conditions.

In an embodiment, the sweat solution was prepared as indicated in AATCC method 15-2009 “Colorfastness to Perspiration”. The pH of the prepared solution varied between 4.0 and 8.5. In accordance with its composition, the sweat solution is also regarded as electrolyte solution.

In a further embodiment, fluorescence emission at 481 nm (excitation at 295 nm) was recorded and the dissociation constant (Kd) calculated. The association constant (Ka) was calculated by formula Kd=1/Ka.

In an embodiment, the release of fragrance was quantified by gas chromatography-mass spectrometry (GC-MS). Increased concentrations of fragrance were used in different vials and the fragrance in headspace quantified performing the calibration curve (area of peak vs fragrance concentration). The fragrance was incubated with the odorant-binding protein at 37° C. Sweat solution was added and the fragrance release determined after several periods of time (0.5-24 h) of perspiration exposition.

In an embodiment, as comparative data, porcine odorant-binding protein was fused with a spacer GQ₂₀ and a carbohydrate-binding module (CBM), as previously reported.²⁵ Through the fusion of CBMN1 (PDB ID 1ULP) of endoglucanase C from Cellulomonas fimi, the OBP has a specific affinity to cotton. The modified protein showed a high association constant (Ka=4.17±0.05 μM) that decreased for Ka=3.16±0.02 UM when a sweat solution was added. Native OBP-I (SEQ ID No. 1) and OBP::GQ₂₀::KP (SEQ ID NO. 21) showed an association constant very similar (Ka=4.00±0.03 μM) to the value obtained for OBP::GQ₂₀::CBM (SEQ ID NO. 22, Ka=4.17±0.05). However, the addition of the sweat solution had a remarkable effect in the constant of affinity of the native protein (Ka=0.20±0.02 μM) and of the OBP::GQ₂₀::KP (Ka=0.59±0.01 μM). A high reduction of affinity was observed, 20× using native OBP (SEQ ID NO. 1) and 6.8× using OBP::GQ₂₀::KP (SEQ ID NO. 21), as compared with the value quantified for protein fused with CBM (SEQ ID NO 22), where a reduction of only 1.3× was verified (Ka=3.16±0.02 Table 2). Thus, the release of fragrance by OBP-I and OBP fused with KP is evident in response of perspiration.

Table 2. Affinity constant (Ka) of native OBP-I (SEQ ID NO. 1) and fusion proteins based on OBP, SEQ ID NO. 21 and SEQ ID NO 22. SEQ ID NO 22 was used as comparative data. Values are the mean of 2 independent experiments at 37° C.

TABLE 2
Affinity constant (Ka) of native OBP-I (SEQ ID NO. 1)
and fusion proteins based on OBP, SEQ ID NO. 21 and
SEQ ID NO 22. SEQ ID NO 22 was used as comparative data.
Values are the mean of 2 independent experiments at 37° C.

| | Ka in buffer | Ka in sweat |
|---|---|---|
| Protein | (μM) | (μM) |
| Native porcine OBP | 4.00 ± 0.03 | 0.20 ± 0.02 |
| (OBP-I)-SEQ ID NO 1 | | |
| OBP::GQ₂₀::KP-SEQ ID NO 21 | 4.00 ± 0.04 | 0.59 ± 0.01 |
| OBP::GQ₂₀::CBM | 4.17 ± 0.05 | 3.16 ± 0.02 |
| (fusion protein)²⁵-SEQ ID NO 22 | | |

In an embodiment, the following protein sequences can be incorporated in different substrates in order to release fragrances in the presence of sweat. In this regard, substrates can be selected from a list comprising textiles, fabrics, skin care products, hair care products, among others.

### List of Protein Sequences

The sequences of protein are described by one letter code of amino acids. The code is as follows:

| One | |
|---|---|
| letter | |
| code | Amino acid |
| A | Alanine |
| C | Cysteine |
| D | Aspartic acid |
| E | Glutamic acid |
| F | Phenylalanine |
| G | Glycine |
| H | Histidine |
| I | Isoleucine |
| K | Lysine |
| L | Leucine |
| M | Methionine |
| N | Asparagine |
| P | Proline |
| Q | Glutamine |
| R | Arginine |
| S | Serine |
| T | Threonine |
| V | Valine |
| W | Tryptophan |
| Y | Tyrosine |

Pig OBP (PDB ID 1DZK)
SEQ ID NO. 1
QEPQPEQDPFELSGKWITSYIGSSDLEKIGENAPFQVFMRSIEFDDKESKVYLNFFSKENGICEE
FSLIGTKQEGNTYDVNYAGNNKFVVSYASETALIISNINVDEEGDKTIMTGLLGKGTDIEDQDLE
KFKEVTRENGIPEENIVNIIERDDCPA
Human OBPₗₗₐ (UniProt ID Q9NY56)
SEQ ID NO. 2
MKTLFLGVTLGLAAALSFTLEEEDITGTWYVKAMVVDKDFPEDRRPRKVSPVKVTALGGGNLEAT
FTFMREDRCIQKKILMRKTEEPGKFSAYGGRKLIYLQELPGTDDYVFYCKDQRRGGLRYMGKLVG
RNPNTNLEALEEFKKLVQHKGLSEEDIFMPLQTGSCVLEH
Human OBPllb (UniProt ID Q9NPH6)
SEQ ID NO. 3
MKTLFLGVTLGLAAALSFTLEEEDITGTWYVKAMVVDKDFPEDRRPRKVSPVKVTALGGGKLEAT
FTFMREDRCIQKKILMRKTEEPGKYSAYGGRKLMYLQELPRRDHYIFYCKDQHHGGLLHMGKLVG
RNSDTNREALEEFKKLVQRKGLSEEDIFTPLQTGSCVPEH
Mouse OBP (UniProt ID OBP1A)
SEQ ID NO. 4
MAKFLLLALTFGLAHAAMEGPWKTVAIAADRVDKIERGGELRIYCRSLTCEKECKEMKVTFYVNE
NGQCSLTTITGYLQEDGKTYKTQFQGNNRYKLVDESPENLTFYSENVDRADRKTKLLFILGHGPL
TSEQKEKFAELAEEKGIPAGNIREVLITDYCPE
Mouse OBP2A (UniProt ID Q8K1H9)
SEQ ID NO. 5
MKSLLLTILLLGLVAVLKAQEAPPDDLVDYSGIWYAKAMVHNGTLPSHKIPSIVFPVRIIALEEG
DLETTVVFWNNGHCREFKFVMKKTEEPGKYTAFHNTKVIHVEKTSVNEHYIFYCEGRHNGTSSFG
MGKLMGRDSGENPEAMEEFKNFIKRMNLRLENMFVPEIGDKCVESD
Mouse OBP1B (UniProt ID A2AEP0)
SEQ ID NO. 6
MMVKFLLLALVFGLAHVHAHDHPELQGQWKTTAIMADNIDKIETSGPLELFVREITCDEGCQKMK
VTFYVKQNGQCSLTTVTGYKQEDGKTFKNQYEGENNYKLLKATSENLVFYDENVDRASRKTKLLY
ILGKGEALTHEQKERLTELATQKGIPAGNLRELAHEDTCPE
Rat OBP (PDB ID 3FIQ)
SEQ ID NO. 7
HHENLDISPSEVNGDWRTLYIVADNVEKVAEGGSLRAYFQHMECGDECQELKIIFNVKLDSECQT
HTVVGQKHEDGRYTTDYSGRNYFHVLKKTDDIIFFHNVNVDESGKETNVILVAGKREDLNKAQKQ
ELRKLAEEYNIPNENTQHLVPTDTCNQ
Rat OBP (PDB ID 3ZQ3)
SEQ ID NO. 8
MRGSHHHHHHTDPEEASFERGNLDVDKLNGDWFSIVVASDKREKIEENGSMRVFVQHIDVLENSL
GFTFRIKENGVCTEFSLVADKTAKDGEYFVEYDGENTFTILKTDYDNYVMFHLVNVNNGETFQLM
ELYGRTKDLSSDIKEKFAKLCVAHGITRDNIIDLTKTDRCLQA
Rat OBP (UniProt ID P08937)
SEQ ID NO. 9
MVKFLLIVLALGVSCAHHENLDISPSEVNGDWRTLYIVADNVEKVAEGGSLRAYFQHMECGDECQ
ELKIIFNVKLDSECQTHTVVGQKHEDGRYTTDYSGRNYFHVLKKTDDIIFFHNVNVDESGRRQCD
LVAGKREDLNKAQKQELRKLAEEYNIPNENTQHLVPTDTCNQ
Bovine OBP (PDB ID 1OBP)
SEQ ID NO. 10
AQEEEAEQNLSELSGPWRTVYIGSTNPEKIQENGPFRTYFRELVFDDEKGTVDFYFSVKRDGKWK
NVHVKATKQDDGTYVADYEGQNVFKIVSLSRTHLVAHNINVDKHGQTTELTGLFVKLNVEDEDLE
KFWKLTEDKGIDKKNVVNFLENEDHPHPE
Boar OBP (PDB ID 1GM6)
SEQ ID NO. 11
HKEAGQDVVTSNFDASKIAGEWYSILLASDAKENIEENGSMRVFVEHIRVLDNSSLAFKFQRKVN
GECTDFYAVCDKVGDGVYTVAYYGENKFRLLEVNYSDYVILHLVDVNGDKTFQLMEFYGRKPDVE
PKLKDKFVEICQQYGIIKENIIDLTKIDRCFQLRGSGGVQESSAE
Panda OBP (PDB ID 5NGH)
SEQ ID NO. 12
HEEGNDVRRNFDVSKISGYWYSVLLASDVREKTEENSSMRVFVNHIEVLSNSSLLFNMHIKVDGK
CTEIALVSDKTEKDGEYSVEYDGYNVFRIVETDYTDYIIFHLVNFKEKDSFQMMELSAREPDTSE
EVRKRFVEYCQKHGIVKENIFDLTEVDRCLQARGSEKA
Chinese hamster OBP (Ensembl ID ENSCGRP00015014591.1)
SEQ ID NO. 13
MVKFLLLAFALSVSCAHHKIPEISPSEVDGKWRTLYIGADNTEKVIQGGPLRAYFRHMECSDECQ
TLTITFNTKEEGKCQTHTVVGRKDEDGQYKTGFSGNNDFHVVEKADGIIIFHNVNVDSSGKKTNV
ILVAGKGKSLSKEQKERLENIAKEFDISKENIQHLVPTDTCDQ
Meishan pig OBP (Ensembl ID ENSSSCP00040041163.1)
SEQ ID NO. 14
MKSLLLSLVLGLVCAQEPQPEQDPFVLSGKWITSYIGSSDLEKIGENAPFQVFMRSIEFDDKESK
VYLNFFSKENGICEEFSLIGTKQEGNTYDVNYAGNNKFVVSYASETALIISNINVDEEGDKTIMT
GLLGKGTDIEDQDLEKFKEVTRENGIPEENIVNIIERDDCPAK
Horse OBP (Ensembl ID ENSECAP00000000103.2)
SEQ ID NO. 15
MQILLLSLVLGVVCAVQEPQSETDYSLFSGEWNTIYIGSSNIEKISENGPFRILLRRLDLDSAGD
RIIYTFFLKVNGQCTKISSLAIKTEENTYVCHYAGKNKFEILHLSKTAIIIDIVNEDEGGLVTKM
VALVGMLGDIQKEDIEKFKEVAKEKEIPEENIVNIINIDDCPTSE
Guinea pig OBP ((Ensembl ID ENSCPOP00000016393.2)
SEQ ID NO. 16
MQILLLALTIGLAYAHQTLDPSEINGQWHTISIAADNVEKIGEGGPLRGYFHNLHCYDGCKNIGL
TFYVKLDGNCQRFDVLGAKQEDSDVYVAQYSGTNHFEVIGKKEDAIAFYNHNTDETGKETKMIVV
VARRDSLTEEEQQKLQEVAGEKGIPKDNIRYFRERDTCAQ
Dog OBP ((Ensembl ID ENSCAFP00040020992.1)
SEQ ID NO. 17
MKILLLCLILVLACDAHLPLPNVLTQVSGPWKTLYVSSNNLDKIAENGPFRIYIRRINVDIPRLK
ILFSFFVKVDGECVEKSVEASIGQDNLINAHYAGGNYHQILDVTPNALIGYIVNVDDKGRITKLA
SLVGRGAHVNEEDIAKFKKLSREKGIPEENIIYLGDTDNCPNHE
Tibetan pig OBP (Ensembl ID ENSSSCP00015013912.1)
SEQ ID NO. 18
MKSLLLSLVLGLVCAQEPQPEQDPFELSGKWITSYIGSSDLEKIGENAPFQVFMRSIEFDDKESK
VYLNFFSKENGICEEFSLTGTKQEGNTYDVNYAGNNKFVVSYASETALIIANINVDEEGDKTIMT
GLLGKGTDIEDQDLEKFKEVTRENGIPEENIVNIIERDDCPAK
Cat felis OBP (Ensembl ID ENSFCAP00000053707.1)
SEQ ID NO. 19
RSCVIHLQCLPTGCLFSALHNGLPDGRLPLPDGRLPLPDGRLPLPDSRLPLPDGRLPLPDGRLPL
PDGRLPLPDGRLPLPDGRLPLPDGRLPLPDGRLPLPDGRLPLPEGRLPLPDSHPPLQDNLTQLSG
EWNTLLVAATNVDKISNGPFHGYICKVDVDVTNGTVVFNFSVMMNGRCTEKSAVGTIGRDKFINI
GSMNQNLFNLFSVTSNTIAINVNTRRNTTKAFALLDTNGNIFNIGYDSLGSLIIHTANVDTAGQT
TQVFALLGKRLHPDDNDFAKFRELMRENNIPEENLIDMSKTEKCPKKEKGTNPS
Chimpanzee OBP (Ensembl ID ENSPTRP00000048681.3)
SEQ ID NO. 20
MALLLLSLGLSLITAQEFDPRNVMQRNYNMARVSGVWYSIFMADDLNRIKENGDLRVFVQNIEHL
KNGSLKFDFEYMVQGECVAVVVVCEKTEKNGEYSINYEGQNTVAVSETDYRLFITFHLQNFRNGT
ETHTLALYETCKKYGLGSQNIINLTNKDPCYSKHYRSPPRPPMRE
OBP::GQ₂₀::KP (recombinant protein)
SEQ ID NO. 21
QEPQPEQDPFELSGKWITSYIGSSDLEKIGENAPFQVFMRSIEFDDKESKVYLNFFSKENGICEE
FSLIGTKQEGNTYDVNYAGNNKFVVSYASETALIISNINVDEEGDKTIMTGLLGKGTDIEDQDLE
KFKEVTRENGIPEENIVNIIERDDCPAGQGQGQGQGQGQGQGQGQGQGQGQGQGQGQGQGQGQGQ
GQGGVCGPSPPCITT
OBP::GQ₂₀::CBM (recombinant protein)
SEQ ID NO. 22
QEPQPEQDPFELSGKWITSYIGSSDLEKIGENAPFQVFMRSIEFDDKESKVYLNFFSKENGICEE
FSLIGTKQEGNTYDVNYAGNNKFVVSYASETALIISNINVDEEGDKTIMTGLLGKGTDIEDQDLE
KFKEVTRENGIPEENIVNIIERDDCPAGQGQGQGQGQGQGQGQGQGQGQGQGQGQGQGQGQGQGQ
GQASPIGEGTFDDGPEGWVAYGTDGPLDTSTGALCVAVPAGSAQYGVGVVLNGVAIEEGTTYTLR
YTATASTDVTVRALVGQNGAPYGTVLDTSPALTSEPRQVTETFTASATYPATPAADDPEGQIAFQ
LGGFSADAWTLCLDDVALDSEVEL

### REFERENCES

(1) Flower, D. R. The lipocalin protein family: structure and function. Biochem. J. 1996, 318, 1-14.
(2) Flower, D. R. Beyond the superfamily: the lipocalin receptors. Biochimica et biophysica acta 2000, 1482, 327-336.
(3) Breer, H. Olfactory receptors: molecular basis for recognition and discrimination of odors. Analytical and bioanalytical chemistry 2003, 377 (3), 427-33, DOI: 10.1007/s00216-003-2113-9.
(4) Tegoni, M.; Pelosi, P.; Vincent, F.; Spinelli, S.; Campanacci, V.; Grolli, S.; Ramoni, R.; Cambillau, C. Mammalian odorant binding proteins. Biochimica et biophysica acta 2000, 1482, 229-240.
(5) Bignetti, E.; Cattaneo, P.; Cavaggioni, A.; Damiani, G. The pyrazine-binding protein and olfaction. Comp. Biochem. Physiol. 1988, 90B, 1-5.
(6) Pevsner, J.; Hou, V.; Snowman, A. M.; Snyder, S. H. Odorant-binding protein: characterization of ligand binding. The Journal of biological chemistry 1990, 265 (11), 6118-6125.
(7) Bignetti, E.; Cavaggioni, A.; Pelosi, P.; Persaud, K. C.; Sorbi, R. T.; Tirindelli, R. Purification and characterisation of an odorant-binding protein from cow nasal tissue. Eur. J. Biochem. 1985, 149, 227-231.
(8) Dal Monte, M.; Andreini, I.; Revoltella, R.; Pelosi, P. Purification and characterization of two odorant-binding proteins from nasal tisue of rabbit and pig. Comp Biochem Physiol 1991, 99B (2), 445-451.
(9) Garibotti, M.; Navarrini, A.; Pisanelli, A. M.; Pelosi, P. Three Odorant-binding Proteins from Rabbit Nasal Mucosa. Chemical senses 1997, 22 (4), 383-390.
(10) Lazar, J.; Greenwood, D. R.; Rasmussen, L. E. L.; Prestwich, G. D. Molecular and Functional Characterization of an Odorant Binding Protein of the Asian Elephant, Elephas maximus: Implications for the Role of Lipocalins in Mammalian Olfaction. Biochemistry 2002, 41, 11786-11794.
(11) Pes, D.; Dal Monte, M.; Ganni, M.; Pelosi, P. Isolation of two odorant-binding proteins from mouse nasal tissue. Comp. Biochem. Physiol. 1992, 103B (4), 1011-1017.
(12) Lobel, D.; Jacob, M.; Volkner, M.; Breer, H. Odorant of different chemica classes interact with distinct odorant binding protein subtypes. Chemical senses 2002, 27, 39-44.
(13) Briand, L.; Eloit, C.; Nespoulous, C.; Bezirard, V.; Huet, J.-C.; Henry, C.; Blon, F.; Trotier, D.; Permollet, J.-C. Evidence of an Odorant-Binding Protein in the Human Olfactory Mucus: Location, Structural Characterization, and Odorant-Binding Properties. Biochemistry 2002, 41, 7241-7252.
(14) Pelosi, P. Odorant-Binding Proteins: Structural Aspects. In Annals New York academy of sciences; Olfaction and Taste XII: an international symposium, 1998; pp 281-293.
(15) Spinelli, S.; Ramoni, R.; Grolli, S.; Bonicel, J.; Cambillau, C.; Tegoni, M. The Structure of the Monomeric Porcine Odorant Binding Protein Sheds Light on the Domain Swapping Mechanism. Biochemistry 1998, 37, 7913-7918.
(16) Perduca, M.; Mancia, F.; Del Giorgio, R.; Monaco, H. L. Crystal Structure of a Truncated Formof Porcine Odorant-Binding Protein. Proteins: Structure, Function, and Genetics 2001, 42, 201-209.
(17) Cave, J. W.; Wickiser, J. K.; Mitropoulos, A. N. Progress in the development of olfactory-based bioelectronic chemosensors. Biosensors & bioelectronics 2019, 123, 211-222, DOI: 10.1016/j.bios.2018.08.063.
(18) Pelosi, P.; Mastrogiacomo, R.; lovinella, I.; Tuccori, E.; Persaud, K. C. Structure and biotechnological applications of odorant-binding proteins. Applied microbiology and biotechnology 2014, 98 (1), 61-70, DOI: 10.1007/s00253-013-5383-y.
(19) Mulla, M. Y.; Tuccori, E.; Magliulo, M.; Lattanzi, G.; Palazzo, G.; Persaud, K.; Torsi, L. Capacitance-modulated transistor detects odorant binding protein chiral interactions. Nature communications 2015, 6, 6010, DOI: 10.1038/ncomms7010.
(20) Paolini, S.; Tanfani, F.; Fini, C.; Bertoli, E.; Pelosi, P. Porcine odorant-binding protein: structural stability and ligand afinities measured by Fourier-transform infrared spectroscopy and fluorescence spectroscopy. Biochimica et biophysica acta 1999, 1431, 179-188.
(21) Sorokowska, A.; Sorokowski, P.; Szmajke, A. Does Personality Smell? Accuracy of Personality Assessments Based on Body Odour. European Journal of Personality 2012, 26 (5), 496-503, DOI: 10.1002/per.848.
(22) Ozeki, C.; Moro, O. A study of the suppression of body odour in elderly subjects by anti-fungal agents. International journal of cosmetic science 2016, 38 (3), 312-8, DOI: 10.1111/ics.12295.
(23) Di Pietrantonio, F.; Cannata, D.; Benetti, M.; Verona, E.; Varriale, A.; Staiano, M.; D'Auria, S. Detection of odorant molecules via surface acoustic wave biosensor array based on odorant-binding proteins. Biosensors & bioelectronics 2013, 41, 328-34, DOI: 10.1016/j.bios.2012.08.046.
(24) Sankaran, S.; Khot, L. R.; Panigrahi, S. Biology and applications of olfactory sensing system: A review. Sensors and Actuators B: Chemical 2012, 171-172, 1-17, DOI: 10.1016/j.snb.2012.03.029.
(25) Goncalves, F.; Ribeiro, A.; Silva, C.; Cavaco-Paulo, A. Release of Fragrances from Cotton Functionalized with Carbohydrate-Binding Module Proteins. ACS Appl Mater Interfaces 2019, DOI: 10.1021/acsami.9b08191.
(26) Kozlowski, L. P. IPC-Isoelectric Point Calculator. Biology direct 2016, 11 (1), 55, DOI: 10.1186/s13062-016-0159-9.
(27) Cennamo, N.; Di Giovanni, S.; Varriale, A.; Staiano, M.; Di Pietrantonio, F.; Notargiacomo, A.; Zeni, L.; D'Auria, S. Easy to Use Plastic Optical Fiber-Based Biosensor for Detection of PloS Butanal. one 2015, 10 (3), e0116770, DOI: 10.1371/journal.pone.0116770.
(28) Capo, A.; Pennacchio, A.; Varriale, A.; D'Auria, S.; Staiano, M. The porcine odorant-binding protein as molecular probe for benzene detection. PloS one 2018, 13 (9), e0202630, DOI: 10.1371/journal.pone.0202630.
(29) Vincent, F.; Ramoni, R.; Spinelli, S.; Grolli, S.; Tegoni, M.; Cambillau, C. Crystal structures of bovine odorant-binding protein in complex with odorant molecules. European journal of biochemistry 2004, 271 (19), 3832-42, DOI: 10.1111/j.1432-1033.2004.04315.x.
(30) Goncalves, F.; Castro, T. G.; Nogueira, E.; Pires, R.; Silva, C.; Ribeiro, A.; Cavaco-Paulo, A. OBP fused with cell-penetrating peptides promotes liposomal transduction. Colloids and surfaces. B, Biointerfaces 2018, 161, 645-653, DOI: 10.1016/j.colsurfb.2017.11.026.
(31) Malpeli, G.; Folli, C.; Cavazzini, D.; Sartori, G.; Berti, R. Purification and Fluorescent Titration of Cellular Retinol-Binding Protein. In Methods in Molecular Biology; Redfern, C. P. F., Ed.; 1998; pp 111-122.
(32) Nogueira, E.; Mangialavori, I. C.; Loureiro, A.; Azoia, N. G.; Sárria, M. P.; Nogueira, P.; Freitas, J.; Harmark, J.; Shimanovich, U.; Rollet, A.; Lacroix, G.; Bernardes, G. J. L.; Guebitz, G.; Hebert, H.; Moreira, A.; Carmo, A. M.; Rossi, J. P. F. C.; Gomes, A. C.; Preto, A.; Cavaco-Paulo, A. Peptide anchor for folate-targeted liposomal delivery. Biomacromolecules 2015, 16 (9), 2904-2910, DOI: 10.1021/acs.biomac.5b00823.
(33) Gonçalves, F.; Silva, C.; Ribeiro, A.; Cavaco-Paulo, A. 1-Aminoanthracene Transduction into Liposomes Driven by Odorant-Binding Protein Proximity. ACS Applied Materials & Interfaces 2018, DOI: 10.1021/acsami.8b10158.
(34) Goncalves, F.; Castro, T. G.; Azoia, N. G.; Ribeiro, A.; Silva, C.; Cavaco-Paulo, A. Two Engineered OBPs with opposite temperature-dependent affinities towards 1-aminoanthracene. Scientific reports 2018, 8 (1), 14844, DOI: 10.1038/s41598-018-33085-8.

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

1-22. (canceled)

23. A composition for hair care comprising a protein comprising an amino acid sequence at least 90% identical to any one of: SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15. SEQ. ID NO. 16, SEQ. ID NO. 17, SEQ. ID NO. 18, SEQ. ID NO. 19, SEQ. ID NO. 20, SEQ. ID NO. 21; and an active agent selected from a list consisting of: a deodorizing agent, a natural essence, a fragrance, a moisturizing agent, or mixtures thereof.

24. The composition according to claim 23, wherein the protein comprises the amino acid sequence at least 95% identical to SEQ ID NO: 10-21.

25. The composition according to claim 23, wherein the protein comprises the amino acid sequence at least identical to SEQ ID NO: 10-21.

26. The composition according to claim 23, wherein the protein comprises the amino acid sequence at least identical to SEQ ID NO: 21.

27. The composition according to claim 23, wherein the protein comprises the amino acid sequence at least identical to SEQ ID NO: 10.

28. The composition according to claim 23, wherein the protein comprises the amino acid sequence at least identical to SEQ ID NO: 11.

29. The composition according to claim 23, wherein the protein comprises the amino acid sequence at least identical to SEQ ID NO: 12.

30. The composition according to claim 23, wherein the protein comprises the amino acid sequence at least identical to SEQ ID NO: 13.

31. The composition according to claim 23, wherein the protein comprises the amino acid sequence at least identical to SEQ ID NO: 14.

32. The composition according to claim 23, wherein the protein comprises the amino acid sequence at least identical to SEQ ID NO: 15.

33. The composition according to claim 23, wherein the protein comprises the amino acid sequence at least identical to SEQ ID NO: 16.

34. The composition according to claim 23, wherein the protein comprises the amino acid sequence at least identical to SEQ ID NO: 17.

35. The composition according to claim 23, wherein the protein comprises the amino acid sequence at least identical to SEQ ID NO: 18.

36. The composition according to claim 23, wherein the protein comprises the amino acid sequence at least identical to SEQ ID NO: 19.

37. The composition according to claim 23, wherein the protein comprises the amino acid sequence at least identical to SEQ ID NO: 20.

38. The composition according to claim 23, wherein the composition is a hair shampoo.

39. The composition according to claim 23, wherein the composition is a as a deodorant agent.

40. The composition according to claim 23, comprising 0.1 μM to 2 M of protein.

41. The composition according to claim 23, further comprising glycerol, erythritol, arabitol, sorbitol, mannitol, xylitol, mannitol, glucosylglycerol, glucose, fructose, sucrose, trehalose, isofluoroside, dextrans, levans, polyethylene glycol, salts of chloride, citrate, sulfates, acetate, or phosphates, or mixtures thereof.

42. The composition according to claim 23, further comprising a buffer solution.


SEQUENCE LISTING (22 sequences)

SEQ ID NO: 1 - protein, 157 aa, Sus scrofa
  REGION 1..157: note = Pig OBP (PDB ID 1DZK)
QEPQPEQDPFELSGKWITSYIGSSDLEKIGENAPFQVFMRSIEFDDKESKVYLNFFSKENGICEEFSLIGTKQEGNTYDVNYAGNNKFVVSYASETALIISNINVDEEGDKTIMTGLLGKGTDIEDQDLEKFKEVTRENGIPEENIVNIIERDDCPA

SEQ ID NO: 2 - protein, 170 aa, Homo sapiens
  REGION 1..170: note = Human OBPIIa (UniProt ID Q9NY56)
MKTLFLGVTLGLAAALSFTLEEEDITGTWYVKAMVVDKDFPEDRRPRKVSPVKVTALGGGNLEATFTFMREDRCIQKKILMRKTEEPGKFSAYGGRKLIYLQELPGTDDYVFYCKDQRRGGLRYMGKLVGRNPNTNLEALEEFKKLVQHKGLSEEDIFMPLQTGSCVLEH

SEQ ID NO: 3 - protein, 170 aa, Homo sapiens
  REGION 1..170: note = Human OBPIIb (UniProt ID Q9NPH6)
MKTLFLGVTLGLAAALSFTLEEEDITGTWYVKAMVVDKDFPEDRRPRKVSPVKVTALGGGKLEATFTFMREDRCIQKKILMRKTEEPGKYSAYGGRKLMYLQELPRRDHYIFYCKDQHHGGLLHMGKLVGRNSDTNREALEEFKKLVQRKGLSEEDIFTPLQTGSCVPEH

SEQ ID NO: 4 - protein, 163 aa, Mus musculus
  REGION 1..163: note = Mouse OBP (UniProt ID OBP1A)
MAKFLLLALTFGLAHAAMEGPWKTVAIAADRVDKIERGGELRIYCRSLTCEKECKEMKVTFYVNENGQCSLTTITGYLQEDGKTYKTQFQGNNRYKLVDESPENLTFYSENVDRADRKTKLLFILGHGPLTSEQKEKFAELAEEKGIPAGNIREVLITDYCPE

SEQ ID NO: 5 - protein, 176 aa, Mus musculus
  REGION 1..176: note = Mouse OBP2A (UniProt ID Q8K1H9)
MKSLLLTILLLGLVAVLKAQEAPPDDLVDYSGIWYAKAMVHNGTLPSHKIPSIVFPVRIIALEEGDLETTVVFWNNGHCREFKFVMKKTEEPGKYTAFHNTKVIHVEKTSVNEHYIFYCEGRHNGTSSFGMGKLMGRDSGENPEAMEEFKNFIKRMNLRLENMFVPEIGDKCVESD

SEQ ID NO: 6 - protein, 171 aa, Mus musculus
  REGION 1..171: note = Mouse OBP1B (UniProt ID A2AEP0)
MMVKFLLLALVFGLAHVHAHDHPELQGQWKTTAIMADNIDKIETSGPLELFVREITCDEGCQKMKVTFYVKQNGQCSLTTVTGYKQEDGKTFKNQYEGENNYKLLKATSENLVFYDENVDRASRKTKLLYILGKGEALTHEQKERLTELATQKGIPAGNLRELAHEDTCPE

SEQ ID NO: 7 - protein, 157 aa, Rattus rattus
  REGION 1..157: note = Rat OBP (PDB ID 3FIQ)
HHENLDISPSEVNGDWRTLYIVADNVEKVAEGGSLRAYFQHMECGDECQELKIIFNVKLDSECQTHTVVGQKHEDGRYTTDYSGRNYFHVLKKTDDIIFFHNVNVDESGKETNVILVAGKREDLNKAQKQELRKLAEEYNIPNENTQHLVPTDTCNQ

SEQ ID NO: 8 - protein, 173 aa, Rattus rattus
  REGION 1..173: note = Rat OBP (PDB ID 3ZQ3)
MRGSHHHHHHTDPEEASFERGNLDVDKLNGDWFSIVVASDKREKIEENGSMRVFVQHIDVLENSLGFTFRIKENGVCTEFSLVADKTAKDGEYFVEYDGENTFTILKTDYDNYVMFHLVNVNNGETFQLMELYGRTKDLSSDIKEKFAKLCVAHGITRDNIIDLTKTDRCLQA

SEQ ID NO: 9 - protein, 172 aa, Rattus rattus
  REGION 1..172: note = Rat OBP (UniProt ID P08937)
MVKFLLIVLALGVSCAHHENLDISPSEVNGDWRTLYIVADNVEKVAEGGSLRAYFQHMECGDECQELKIIFNVKLDSECQTHTVVGQKHEDGRYTTDYSGRNYFHVLKKTDDIIFFHNVNVDESGRRQCDLVAGKREDLNKAQKQELRKLAEEYNIPNENTQHLVPTDTCNQ

SEQ ID NO: 10 - protein, 159 aa, Bos gaurus
  REGION 1..159: note = Bovine OBP (PDB ID 1OBP)
AQEEEAEQNLSELSGPWRTVYIGSTNPEKIQENGPFRTYFRELVFDDEKGTVDFYFSVKRDGKWKNVHVKATKQDDGTYVADYEGQNVFKIVSLSRTHLVAHNINVDKHGQTTELTGLFVKLNVEDEDLEKFWKLTEDKGIDKKNVVNFLENEDHPHPE

SEQ ID NO: 11 - protein, 175 aa, Sus scrofa
  REGION 1..175: note = Boar OBP (PDB ID 1GM6)
HKEAGQDVVTSNFDASKIAGEWYSILLASDAKENIEENGSMRVFVEHIRVLDNSSLAFKFQRKVNGECTDFYAVCDKVGDGVYTVAYYGENKFRLLEVNYSDYVILHLVDVNGDKTFQLMEFYGRKPDVEPKLKDKFVEICQQYGIIKENIIDLTKIDRCFQLRGSGGVQESSAE

SEQ ID NO: 12 - protein, 168 aa, Ailuropoda melanoleuca
  REGION 1..168: note = Panda OBP (PDB ID 5NGH)
HEEGNDVRRNFDVSKISGYWYSVLLASDVREKTEENSSMRVFVNHIEVLSNSSLLFNMHIKVDGKCTEIALVSDKTEKDGEYSVEYDGYNVFRIVETDYTDYIIFHLVNFKEKDSFQMMELSAREPDTSEEVRKRFVEYCQKHGIVKENIFDLTEVDRCLQARGSEKA

SEQ ID NO: 13 - protein, 173 aa, Cricetulus griseus
  REGION 1..173: note = Chinese hamster OBP (Ensembl ID ENSCGRP00015014591.1)
MVKFLLLAFALSVSCAHHKIPEISPSEVDGKWRTLYIGADNTEKVIQGGPLRAYFRHMECSDECQTLTITFNTKEEGKCQTHTVVGRKDEDGQYKTGFSGNNDFHVVEKADGIIIFHNVNVDSSGKKTNVILVAGKGKSLSKEQKERLENIAKEFDISKENIQHLVPTDTCDQ

SEQ ID NO: 14 - protein, 173 aa, Sus scrofa
  REGION 1..173: note = Meishan pig OBP (Ensembl ID ENSSSCP00040041163.1)
MKSLLLSLVLGLVCAQEPQPEQDPFVLSGKWITSYIGSSDLEKIGENAPFQVFMRSIEFDDKESKVYLNFFSKENGICEEFSLIGTKQEGNTYDVNYAGNNKFVVSYASETALIISNINVDEEGDKTIMTGLLGKGTDIEDQDLEKFKEVTRENGIPEENIVNIIERDDCPAK

SEQ ID NO: 15 - protein, 175 aa, Equus caballus
  REGION 1..175: note = Horse OBP (Ensembl ID ENSECAP00000000103.2)
MQILLLSLVLGVVCAVQEPQSETDYSLFSGEWNTIYIGSSNIEKISENGPFRILLRRLDLDSAGDRIIYTFFLKVNGQCTKISSLAIKTEENTYVCHYAGKNKFEILHLSKTAIIIDIVNEDEGGLVTKMVALVGMLGDIQKEDIEKFKEVAKEKEIPEENIVNIINIDDCPTSE

SEQ ID NO: 16 - protein, 170 aa, Cavia porcellus
  REGION 1..170: note = Guinea pig OBP ((Ensembl ID ENSCPOP00000016393.2)
MQILLLALTIGLAYAHQTLDPSEINGQWHTISIAADNVEKIGEGGPLRGYFHNLHCYDGCKNIGLTFYVKLDGNCQRFDVLGAKQEDSDVYVAQYSGTNHFEVIGKKEDAIAFYNHNTDETGKETKMIVVVARRDSLTEEEQQKLQEVAGEKGIPKDNIRYFRERDTCAQ

SEQ ID NO: 17 - protein, 174 aa, Canis lupus
  REGION 1..174: note = Dog OBP ((Ensembl ID ENSCAFP00040020992.1)
MKILLLCLILVLACDAHLPLPNVLTQVSGPWKTLYVSSNNLDKIAENGPFRIYIRRINVDIPRLKILFSFFVKVDGECVEKSVEASIGQDNLINAHYAGGNYHQILDVTPNALIGYIVNVDDKGRITKLASLVGRGAHVNEEDIAKFKKLSREKGIPEENIIYLGDTDNCPNHE

SEQ ID NO: 18 - protein, 173 aa, Sus scrofa
  REGION 1..173: note = Tibetan pig OBP (Ensembl ID ENSSSCP00015013912.1)
MKSLLLSLVLGLVCAQEPQPEQDPFELSGKWITSYIGSSDLEKIGENAPFQVFMRSIEFDDKESKVYLNFFSKENGICEEFSLTGTKQEGNTYDVNYAGNNKFVVSYASETALIIANINVDEEGDKTIMTGLLGKGTDIEDQDLEKFKEVTRENGIPEENIVNIIERDDCPAK

SEQ ID NO: 19 - protein, 314 aa, Sus scrofa
  REGION 1..314: note = Cat felis OBP (Ensembl ID ENSFCAP00000053707.1)
RSCVIHLQCLPTGCLFSALHNGLPDGRLPLPDGRLPLPDGRLPLPDSRLPLPDGRLPLPDGRLPLPDGRLPLPDGRLPLPDGRLPLPDGRLPLPDGRLPLPDGRLPLPEGRLPLPDSHPPLQDNLTQLSGEWNTLLVAATNVDKISNGPFHGYICKVDVDVTNGTVVFNFSVMMNGRCTEKSAVGTIGRDKFINIGSMNQNLFNLFSVTSNTIAINVNTRRNTTKAFALLDTNGNIFNIGYDSLGSLIIHTANVDTAGQTTQVFALLGKRLHPDDNDFAKFRELMRENNIPEENLIDMSKTEKCPKKEKGTNPS

SEQ ID NO: 20 - protein, 175 aa, Pan troglodytes
  REGION 1..175: note = Chimpanzee OBP (Ensembl ID ENSPTRP00000048681.3)
MALLLLSLGLSLITAQEFDPRNVMQRNYNMARVSGVWYSIFMADDLNRIKENGDLRVFVQNIEHLKNGSLKFDFEYMVQGECVAVVVVCEKTEKNGEYSINYEGQNTVAVSETDYRLFITFHLQNFRNGTETHTLALYETCKKYGLGSQNIINLTNKDPCYSKHYRSPPRPPMRE

SEQ ID NO: 21 - protein, 210 aa, synthetic construct
  REGION 1..210: note = OBP::GQ20::KP (recombinant protein)
QEPQPEQDPFELSGKWITSYIGSSDLEKIGENAPFQVFMRSIEFDDKESKVYLNFFSKENGICEEFSLIGTKQEGNTYDVNYAGNNKFVVSYASETALIISNINVDEEGDKTIMTGLLGKGTDIEDQDLEKFKEVTRENGIPEENIVNIIERDDCPAGQGQGQGQGQGQGQGQGQGQGQGQGQGQGQGQGQGQGQGQGGVCGPSPPCITT

SEQ ID NO: 22 - protein, 349 aa, synthetic construct
  REGION 1..349: note = OBP::GQ20::CBM (recombinant protein)
QEPQPEQDPFELSGKWITSYIGSSDLEKIGENAPFQVFMRSIEFDDKESKVYLNFFSKENGICEEFSLIGTKQEGNTYDVNYAGNNKFVVSYASETALIISNINVDEEGDKTIMTGLLGKGTDIEDQDLEKFKEVTRENGIPEENIVNIIERDDCPAGQGQGQGQGQGQGQGQGQGQGQGQGQGQGQGQGQGQGQGQASPIGEGTFDDGPEGWVAYGTDGPLDTSTGALCVAVPAGSAQYGVGVVLNGVAIEEGTTYTLRYTATASTDVTVRALVGQNGAPYGTVLDTSPALTSEPRQVTETFTASATYPATPAADDPEGQIAFQLGGFSADAWTLCLDDVALDSEVEL